(19) United States
(12) Patent Application Publication
(10) Pub. No.: US 2025/0331797 A1
(43) Pub. Date: Oct. 30, 2025

(54) METHOD FOR ACQUIRING BONE DENSITY, X-RAY IMAGING SYSTEM, AND STORAGE MEDIUM

(71) Applicant: GE Precision Healthcare LLC, Waukesha, WI (US)

(72) Inventors: Buer Qi, Beijing (CN); Hongnan Ye, Beijing (CN); Yannan Huang, Beijing (CN); Mingtao Hu, Beijing (CN)

(21) Appl. No.: 19/195,442

(22) Filed: Apr. 30, 2025

(30) Foreign Application Priority Data
Apr. 30, 2024 (CN) 202410543668.4

(51) Int. Cl. A61B 6/50; A61B 6/00; G06T 7/00; G16H 10/60

(52) U.S. Cl. CPC A61B 6/505; A61B 6/5294; G06T 7/0014; G16H 10/60; G06T 2207/30008

(57) ABSTRACT

The present application provides a method for acquiring bone density, an X-ray imaging system, and a storage medium. The method for acquiring bone density includes acquiring at least one X-ray image of a subject under examination using an X-ray imaging system, wherein the at least one X-ray image is a raw image or a medical image following image processing, and on the basis of a trained learning network, performing processing on the at least one X-ray image, and at least one of position of bone with abnormality, probability of abnormality, and prompt of abnormality, the result including at least one of T-score and classification of bone density, and the abnormality denoting that the T-score exceeds a threshold value range.

### CROSS-REFERENCE TO RELATED APPLICATIONS

The present application claims priority to Chinese Application No. 202410543668.4, filed on Apr. 30, 2024, the entire contents of which is hereby incorporated by reference.

### TECHNICAL FIELD

The present invention relates to medical imaging technology, and more specifically, to an X-ray image-based method for acquiring bone density, an X-ray imaging system, and a non-transitory computer-readable storage medium.

### BACKGROUND ART

In an X-ray imaging system, radiation from an X-ray source is emitted toward a subject, and the subject under examination is usually a patient in a medical diagnosis application. Some of the radiation passes through the subject under examination and impacts a detector, which is divided into a matrix of discrete elements (e.g., pixels). The detector elements are read to generate an output signal on the basis of the amount or intensity of radiation that impacts each pixel region. The signal can then be processed to generate a medical image that can be displayed for review, and the medical image can be displayed in a display apparatus of the X-ray imaging system.

In general, in order to acquire the bone density of a subject under examination, it is generally necessary to perform measurement by means of a dedicated bone density measuring instrument to acquire the T-score of bone density of the patient. Currently, it is basically necessary for the subject under examination to often undergo physical examination, and capturing a chest radiograph or capturing an X-ray image is an essential item in the physical examination. Therefore, it is desirable to be able to conveniently acquire the bone density of the subject under examination on the basis of the chest radiograph or the X-ray image captured, such that the subject under examination does not have to be subjected to excess radiation exposure.

### SUMMARY OF THE INVENTION

The present invention provides an X-ray image-based method for acquiring bone density, an X-ray imaging system, and a non-transitory computer-readable storage medium.

An exemplary embodiment of the present invention provides an X-ray image-based method for acquiring bone density. The method for acquiring bone density comprises acquiring at least one X-ray image of a subject under examination using an X-ray imaging system, wherein the at least one X-ray image is a raw image or a medical image following image processing, and on the basis of a trained learning network, outputting the result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

An exemplary embodiment of the present invention further provides a non-transitory computer-readable storage medium for storing a computer program, which when executed by a computer, causes the computer to execute the foregoing method for acquiring bone density.

An exemplary embodiment of the present invention further provides an X-ray imaging system. The X-ray imaging system comprises a control apparatus which is capable of executing the foregoing method for acquiring bone density.

An exemplary embodiment of the present invention further provides an X-ray imaging system. The X-ray imaging system comprises an acquisition unit and an image processing unit. The acquisition unit is capable of acquiring at least one X-ray image of a subject under examination using the X-ray imaging system, wherein the at least one X-ray image is a raw image or a medical image following image processing, and the image processing unit is capable of, on the basis of a trained learning network, outputting the result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of an X-ray imaging system according to some embodiments of the present invention;

FIG. 2 is a schematic diagram of an X-ray imaging system according to some other embodiments of the present invention;

FIG. 3 is a schematic diagram of a control apparatus according to some embodiments of the present invention;

FIG. 4 is a schematic diagram of a control apparatus according to some other embodiments of the present invention;

FIG. 5 is a schematic diagram of a control apparatus according to some further embodiments of the present invention;

FIG. 6 is a schematic diagram of a learning network according to some embodiments of the present invention;

FIG. 7 is a schematic diagram of an X-ray image bearing an indication sign according to some embodiments of the present invention;

FIG. 8 is a schematic diagram of an X-ray image bearing an indication sign according to some other embodiments of the present invention;

FIG. 9 is a flow chart of a method for acquiring bone density according to some embodiments of the present invention; and

FIG. 10 is a flow chart of a method for acquiring bone density according to some other embodiments of the present invention.

### DETAILED DESCRIPTION OF SPECIFIC IMPLEMENTATIONS

Specific implementations of the present invention will be described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

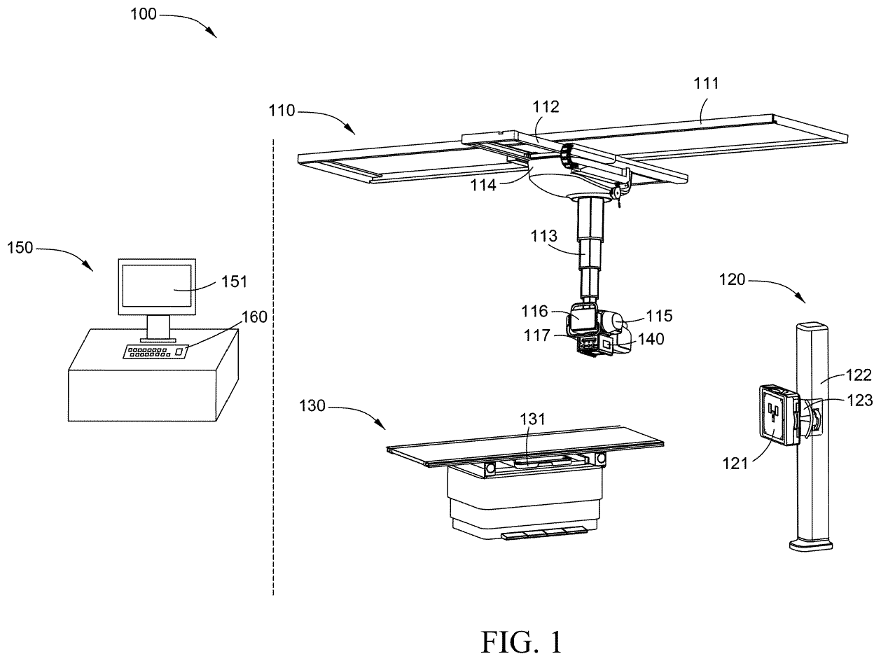

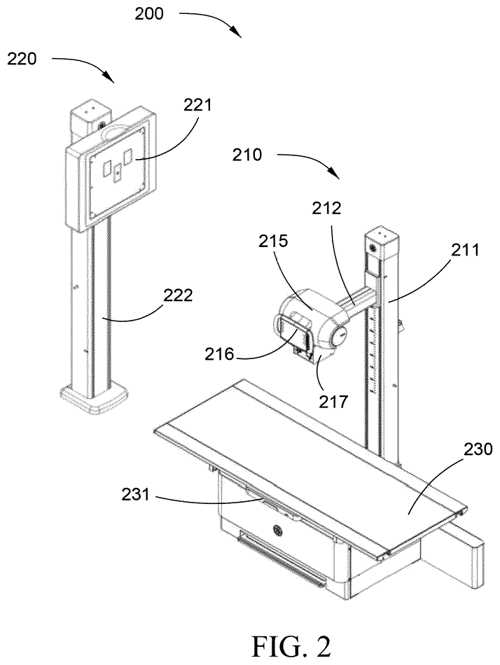

FIG. 1 illustrates an X-ray imaging system 100 according to some embodiments of the present invention, and FIG. 2 illustrates an X-ray imaging system 200 according to some other embodiments of the present invention. As shown in FIG. 1, the X-ray imaging system 100 includes a suspension apparatus 110, a wall stand apparatus 120, and an examination table apparatus 130. The suspension apparatus 110 includes a longitudinal guide rail 111, a transverse guide rail 112, a telescopic cylinder 113, a sliding member 114, a tube assembly 115, and a tube control apparatus 116.

Although the present application is described by taking the suspension type X-ray imaging system shown in FIG. 1 as an example, the method and the apparatus for acquiring bone density in the present application may also be applied to a ground rail type X-ray imaging system and/or a mobile X-ray imaging system shown in FIG. 2. Specifically, an X-ray source may be mounted on a transverse arm of the ground rail type. That is, the transverse arm on which the X-ray source is mounted is, by means of the wall stand, mounted in a rail on the ground, and the X-ray source can move along the rail, the wall stand, and the transverse arm. Of course, the X-ray source may also be, by means of a telescopic arm, mounted on a mobile cart.

For case of description, in the present application, the x-axis, y-axis, and z-axis are defined as the x-axis and y-axis being located in the horizontal plane and perpendicular to one another, and the z-axis being perpendicular to the horizontal plane. Specifically, the direction in which a longitudinal guide rail 111 is located is defined as the x-axis, the direction in which a transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, the longitudinal guide rail 111 being mounted on a ceiling and the transverse guide rail 112 being mounted on the longitudinal guide rail 111. The telescopic cylinder 113 is configured to carry the tube assembly 115.

The sliding member 114 is provided between the transverse guide rail 112 and the telescopic cylinder 113. The sliding member 114 may include components such as a rotating shaft, a motor, and a reel. The motor can drive the reel to rotate around the rotating shaft, which in turn drives the telescopic cylinder 113 to move along the z-axis and/or slide relative to the transverse guide rail. The sliding member 114 is capable of sliding relative to the transverse guide rail 112, i.e., the sliding member 114 is capable of driving the telescopic cylinder 113 and/or the tube assembly 115 to move in the y-axis direction. Further, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of cylinders having different inner diameters, and the plurality of cylinders can be sleeved, sequentially from bottom to top, in the cylinder located thereabove, thereby achieving telescoping, and the telescopic cylinder 113 can be telescopic (or movable) in the vertical direction, i.e., the telescopic cylinder 113 can drive the tube assembly 115 to move along the z-axis direction. The lower end of the telescopic cylinder 113 is further provided with a rotating part, and the rotating part can drive the tube assembly 115 to rotate.

Specifically, the X-ray source and a collimator 117 are provided within the tube assembly 115 and the collimator 117 is typically mounted below the X-ray source. The size of an aperture of the collimator 117 dictates the irradiation range of X-ray, namely the size of the area of an exposure field of view (FOV). X-rays can pass through the opening of the collimator to a region of interest (ROI) of the subject under examination, and other X-rays are absorbed by the shutters to prevent the subject under examination from absorbing an excess unnecessary dose.

In some embodiments, the X-ray imaging system 100 further includes a camera unit 140, and the camera unit 140 is aligned with the detector so as to be configured to acquire a real-time camera image of the subject under examination. In addition, the camera is able to acquire an image of the detector, etc. Specifically, the camera unit 140 is mounted on the suspension apparatus 110, and further, on the side of the collimator 117.

The camera unit 140 may include one or more cameras, for example, a digital camera, an analog camera, etc., or a depth camera, an infrared camera, or an ultraviolet camera, etc., or a 3D camera, a 3D scanner, etc., or a red, green, and blue (RGB) sensor, an RGB depth (RGB-D) sensor, or other devices that can capture color image data of a target subject. In some embodiments, the camera unit 140 is further provided with a control module that can control the rotation of the camera unit to adjust the capture range of the camera unit. In other embodiments, the camera unit is a panoramic camera that can capture an image of the entire body of the subject under examination.

The camera unit 140 can acquire depth information or a depth image of the subject under examination. Typically, the depth information is calculated from a 3D point cloud that is acquired by the camera. In addition, the real-time optical image can be used to acquire at least one of the thickness, height, position, body position, pose, etc. of the subject under examination.

In some embodiments, the camera unit 140 may also be a camera unit that is mounted in a fixed position, or fixed in any other way in a scan room. In some embodiments, the optical image acquired by the camera unit 140 is not limited to a single optical image, but may also include a dynamic real-time video stream, i.e., a series of real-time optical images.

The tube control apparatus (console) 116 is mounted on the tube assembly 115. The tube control apparatus 116 includes user interfaces such as a display screen and a control button so as to be configured to perform pre-capturing preparations, such as patient selection, protocol selection, positioning, etc.

The movement of the suspension apparatus 110 includes the movement of the tube assembly along the x-axis, y-axis, and z-axis, as well as the rotation of the tube assembly in the horizontal plane (the axis of rotation is parallel to or overlaps with the z-axis) and in the vertical plane (the axis of rotation is parallel to the y-axis). In the above motion, a motor is usually used to drive a rotating shaft which in turn drives corresponding components to rotate in order to achieve the corresponding movement or rotation, and the corresponding control components are generally mounted in the sliding member 114. The X-ray imaging system further includes a motion control apparatus (not shown in the figures) that is capable of controlling the movement of the suspension apparatus 110, and furthermore, the motion control apparatus is capable of receiving a control signal to control the corresponding component to move accordingly to drive the tube assembly to reach a preset or specified position.

As shown in FIG. 2, the X-ray imaging system 200 includes a floor-standing apparatus 210, a wall stand apparatus 220, and an examination table apparatus 230. The floor apparatus 210 comprises a support column 211, a cantilever 212, and a tube assembly 215. The cantilever 212 is configured to carry the tube assembly 215, and the cantilever 212 is mounted on the support column 211.

For case of description, in the present application, the x-axis, y-axis, and z-axis are defined as the x-axis and y-axis being located in a horizontal plane and perpendicular to one another, and the z-axis being perpendicular to the horizontal plane. Specifically, the extension direction of the cantilever 212 or the width direction of the examination table apparatus is defined as the x-axis, the direction in the horizontal plane which is perpendicular to the extension direction of the cantilever or the length direction of the examination table apparatus is defined as the y-axis, and the extension direction of the support column 211 is defined as the z-axis direction. The z-axis direction is namely a vertical direction.

The floor apparatus 210 further includes a guide rail mounted on the floor. The guide rail is disposed along the y-axis direction, and the support column 211 moves along the guide rail, i.e., along the y-axis direction. In addition, the cantilever 212 is further capable of moving along the vertical direction (i.e., the z-axis direction) relative to the support column 211. In addition, a drive apparatus may further be provided between the tube assembly 215. The drive apparatus may drive the tube assembly 215 to rotate about the x-axis as a central axis.

The tube assembly 215 includes an X-ray source, a beam limiter 217, and a tube control apparatus 216. The beam limiter 217 and the tube control apparatus 216 are substantially similar in structure and function to the collimator 117 and the tube control apparatus 116 in FIG. 1.

As shown in FIGS. 1-2, the wall stand apparatus 120/220 includes a first detector 121/221, a wall stand 122/222, and a connection member 123 (not shown in FIG. 2). The connection member 123 includes a support arm that is vertically connected in the height direction of the wall stand 122/222 and a rotating bracket that is mounted on the support arm, and the first detector 121/221 is mounted on the rotating bracket. The wall stand apparatus 120/220 further includes a detector driving apparatus that is arranged between the rotating bracket and the first detector 121/221, which is driven by the detector driving apparatus to move in a direction parallel to the height direction of the wall stand 122/222 in the plane held by the rotating bracket. The first detector 121/221 can further be rotated relative to the support arm to form an angle relative to the wall stand. The first detector 121/221 has a plate-like structure whose orientation is variable so that the X-ray incident surface can become vertical or horizontal depending on the incident direction of the X-rays.

A second detector 131/230 is included on the examination table apparatus 130/231. The selection or use of the first detector 121/221 and the second detector 131/231 may be determined on the basis of a capture site of a patient and/or a capture protocol, or may be determined on the basis of the position of the subject under examination that is obtained from a camera capture, so as to perform imaging examination in a supine or standing position.

For case of illustration, components such as a display unit located in a control room are omitted in FIG. 2. However, it should be understood by those skilled in the art that the X-ray imaging system shown in FIG. 2 also includes a similar structure. The X-ray imaging system 100/200 further includes a display unit 150. The display unit 150 is operably connected to the camera unit and includes a user interface 151 configured to display the real-time optical image, the X-ray images, the medical image, the information of the subject under examination, an exposure parameter setting interface, an image post-processing interface, etc.

Specifically, the display unit 150 can include any form of display screen, which may be a main display screen that is located in the control room, a display screen of the tube control apparatus 116/216 that is located in the scan room, or a mobile display, such as a tablet, a cell phone, etc.

The X-ray imaging system 100/200 further includes an input unit 160 configured to receive a user operation. The input unit 160 can include an input device such as a touchscreen, a keyboard, a mouse, a voice-activated control apparatus, or any other suitable input device, and a user can input an operation signal/control signal into the control apparatus by means of the input unit 160.

The X-ray imaging system 100/200 further includes a control apparatus (not shown in the figures), which may be a main control apparatus that is located in the control room, or the tube control apparatus, or a mobile or portable control apparatus, or any combination of the foregoing. The control apparatus may include a source control apparatus and a detector control apparatus. The source control apparatus is used to command the X-ray source to emit X-rays for image exposure. The detector control apparatus is used to select a suitable detector among a plurality of detectors, and to coordinate the control of various detector functions, such as automatically selecting a corresponding detector according to the position or pose of the subject under examination. Alternatively, the detector control apparatus may perform various signal processing and filtering functions, specifically, for initial adjustment of a dynamic range, interleaving of digital image data, and the like. In some embodiments, the control apparatus may provide power and timing signals for controlling the operation of the X-ray source and the detector.

In some embodiments, the control apparatus may also be configured to use a digitized signal to reconstruct one or more required images and/or determine useful diagnostic information corresponding to a patient, and the control apparatus may include one or more dedicated processors, graphics processing units, digital signal processors, microcomputers, microcontrollers, application-specific integrated circuits (ASICs), field-programmable gate arrays (FPGAs), or other appropriate processing apparatuses.

Of course, the X-ray imaging system 100/200 may further include other numbers or configurations or forms of control apparatuses. For example, the control apparatus may be local (e.g., co-located with one or more X-ray imaging systems 100, e.g., within the same facility and/or the same local network). In other implementations, the control apparatus may be remote, and thus only accessible by means of a remote connection (for example, by means of the Internet or other available remote access technologies). In a specific implementation, the control apparatus may also be configured in a cloud-like means, and may be accessed and/or used in a means that is substantially similar to the means by which other cloud-based systems are accessed and used.

In some embodiments, the X-ray imaging system 100/200 further includes an operator workstation, the operator workstation allowing the user to receive and evaluate the reconstructed image, and input a control instruction (an operation signal or a control signal). The operator workstation may include a user interface (or user input device) in a certain form of operator interface, such as a keyboard, a mouse, a voice activated control apparatus, or any other suitable input device, such that an operator may input an operation signal/control signal to the control apparatus by means of the user interface.

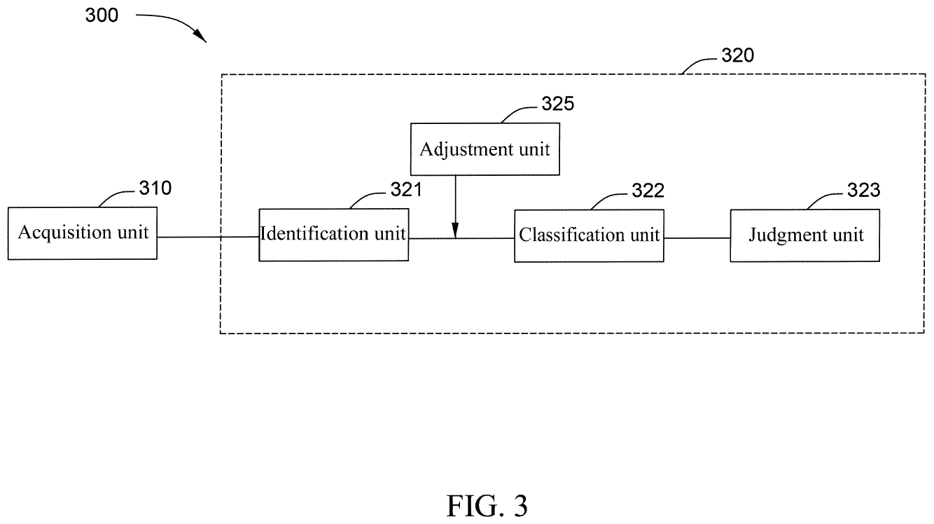

FIG. 3 shows a schematic diagram of a control apparatus 300 according to some embodiments of the present application in order to be able to judge or predict bone density on the basis of an acquired X-ray image. As shown in FIG. 3, the control apparatus 300 includes an acquisition unit 310 and an image processing unit 320.

The acquisition unit 310 is capable of acquiring at least one X-ray image of a subject under examination, wherein the at least one X-ray image is a raw image or a medical image following image processing.

The image processing unit 320 includes a learning network, and is capable of processing the at least one X-ray image on the basis of the trained learning network to output a result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

Specifically, the acquisition unit 310 is capable of being connected to an X-ray source and a detector to control the X-ray source to emit X-rays toward the subject under examination. The detector is capable of detecting an output signal following attenuation by the subject under examination, and is capable of acquiring raw data or raw image on the basis of the output signal. The raw image following image processing is the medical image, and both the raw image and the medical image may be used as the X-ray image. Specifically, the image processing herein is not limited to image post-processing, for example, operations such as image reconstruction, smoothing, noise removal, and artifact removal, but may also include other types of image processing, for example, image stitching or tomosynthesis, etc.

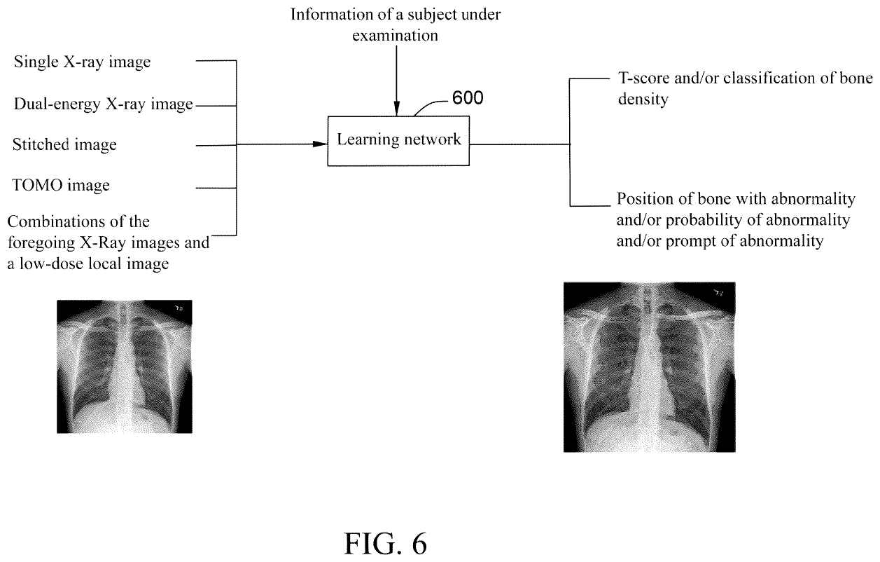

FIG. 6 shows a schematic diagram of a learning network 600 according to some embodiments of the present application. As shown in FIG. 6, the X-ray image includes at least one or a combination of a single X-ray image, a dual-energy X-ray image, a stitched image, a tomographic (TOMO) image, or a combination of at least one of the foregoing and a low-dose local image.

In some embodiments, the input into the learning network may be a single raw image or a single medical image, for example, a chest radiograph, or may be two raw images acquired with different exposure intensities or a medical image following dual energy operation, or may be at least two raw images acquired via an image stitching protocol or a stitched image following image stitching processing (i.e., a medical image), or, of course, may be multiple raw images obtained by means of tomography (TOMO) or a three-dimensional medical image obtained following processing. The foregoing images are all acquired by performing exposure under an original or existing scan protocol of the subject under examination. That is, these images are otherwise to be acquired even if prediction or calculation of bone density is not required to be performed. The subject under examination does not need to be exposed to additional radiation from rays.

Of course, in other embodiments, for some particular subjects under examination, for example, older subjects under examination, a physician or user may also choose to capture an additional low-dose local image under the original scan protocol. The local image is dedicated to a local site of the subject under examination, for example, a site where bone is densely present or a site which is susceptible to osteoporosis or fracture. Specifically, the local image may be for a lumbar vertebra site of the subject under examination. By combining the image obtained under the original scan protocol with the low-dose image of the lumbar vertebra site, prediction or calculation of the bone density of the subject under examination can be better performed. Inputting the combination of the image under the original scan protocol and the low-dose local image into the learning network enables the obtained result of the bone density to be more precise, and the subject under examination does not have to be exposed to excess exposure.

In some embodiments, the image processing unit 320 includes a classification unit 322 and a judgment unit 323.

Specifically, the classification unit 322 performs classification processing on the at least one X-ray image to output a classification of bone density. The judgment unit 323 is capable of further outputting or acquiring at least one of position of bone with abnormality, probability of abnormality, and prompt of abnormality, the abnormality denoting that the T-score exceeds a threshold value range.

Specifically, bone density, short for skeleton mineral density, is an important index of skeleton strength, expressed in grams per cubic centimeter, and is an absolute value. In clinical use of the bone density value, since different bone density testing instruments give different absolute values, the T-score is generally used to judge whether the bone density is normal. The T-score is a relative value, the normal reference value thereof ranging between −1 and +1.

The classification of bone density refers to classification made according to T-score intervals of bone density. For example, bone density may be classified into three classes, including first class, second class, and third class, etc., all of which are classifications made according to a classification method and a classification basis known to a physician or user, or one of ordinary skill in the art. Of course, bone density can be classified into fewer or more classes.

In some embodiments, the outputted T value of bone density is a specific numerical value. The classifications of bone density may be first class, second class, and third class in a Chinese context, or may be level 1, level 2, and level 3 in an English context, or, of course, may be in any other suitable form of representation, for example, outputting expressions such as normal, slightly abnormal, and severely abnormal, etc., which is not limited in the present application.

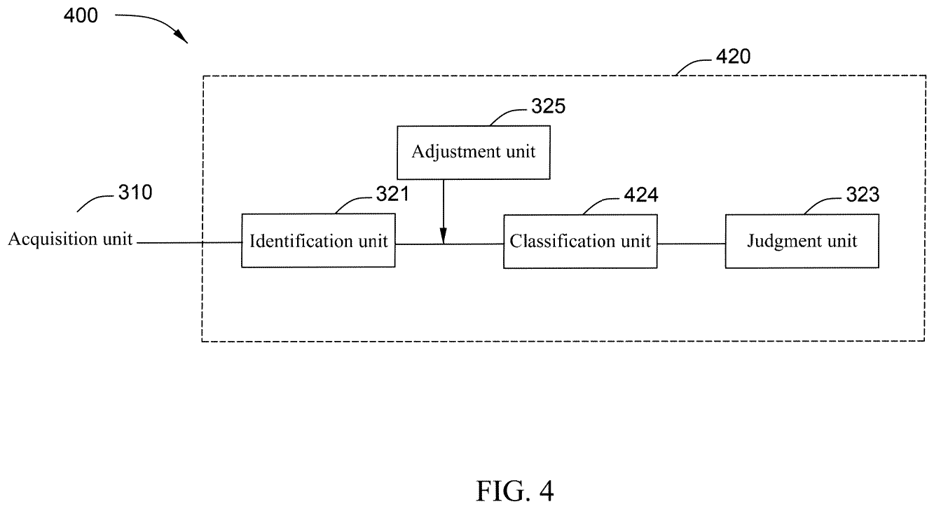

FIG. 4 shows a schematic diagram of a control apparatus 400 according to some other embodiments of the present application, which differs from the control apparatus shown in FIG. 3 in that the image processing unit 420 in the control apparatus shown in FIG. 4 includes a regression unit 424 and a judgment unit 323. The regression unit 424 is capable of performing regression processing on a region of the at least one X-ray image to output a T-score of bone density.

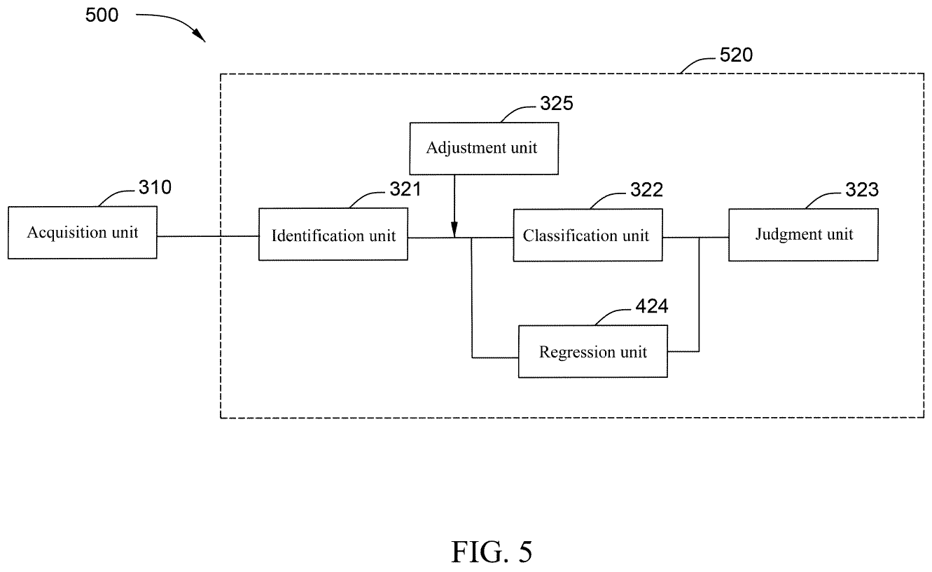

FIG. 5 shows a schematic diagram of a control apparatus 300 according to some further embodiments of the present application. Unlike the control apparatus shown in FIG. 3, the image processing unit 520 in the control apparatus shown in FIG. 5 includes a classification unit 322, a regression unit 424, and a judgment unit 323.

Therefore, the image processing unit, or the learning network, can be customized according to the demand or need of a user. For example, if the user only wants to get the T-score of bone density of the subject under examination, then providing the regression unit in the image processing unit will suffice, if the user wants to get only the classification of bone density of the subject under examination, then providing the classification unit in the image processing unit will suffice, and if the user wants to get both the T-score of bone density and the classification of bone density, the classification unit and the regression unit need to be provided at the same time.

In some embodiments, both the classification unit and the regression unit may be a learning network, which may be obtained by performing training on the basis of training data. When a region of interest of the subject under examination is inputted into the learning network, the learning network can output corresponding T-score and/or classification.

Specifically, for the classification unit, X-ray images of multiple groups of subjects under examination and corresponding classifications of bone density are used as training data. The X-ray images serve as known inputs and the classifications of bone density serve as expected outputs, and the learning network is allowed to learn. When the learning network has been trained, an X-ray image to be judged can be inputted into the learning network, and the corresponding classification of bone density will be obtained. Likewise, for the regression unit, a similar principle applies. The X-ray images serve as known inputs and the T-scores of bone density serve as expected outputs, and the learning network is allowed to learn. When the learning network has been trained, an X-ray image to be judged can be inputted into the learning network, and the corresponding T-score of bone density will be obtained. For the learning network, detailed description will be provided hereinafter.

Referring back to FIG. 3, the judgment unit 323 is capable of outputting or acquiring at least one of position of bone with abnormality, probability of abnormality, and prompt of abnormality. The abnormality denotes that the result of bone density exceeds a threshold value range, and specifically, may be the T-score of bone density exceeding a set threshold value or the classification of bone density exceeding a set limit. Specifically, when the result of bone density exceeds the threshold value range, it is indicated that there is a possibility of osteoporosis or a high probability of osteoporosis at the site in question. Of course, different threshold value ranges may be set to indicate different types of abnormality or degrees of abnormality. In some embodiments, different threshold value ranges may also be set according to information of the subject under examination, for example, according to age or gender. Specifically, for an older subject under examination, the threshold value range may be set to be relatively low, and for a younger subject under examination, the threshold value range may be set to be relatively high. With the customized threshold values, different positions, probabilities and/or prompts of abnormality can be better outputted.

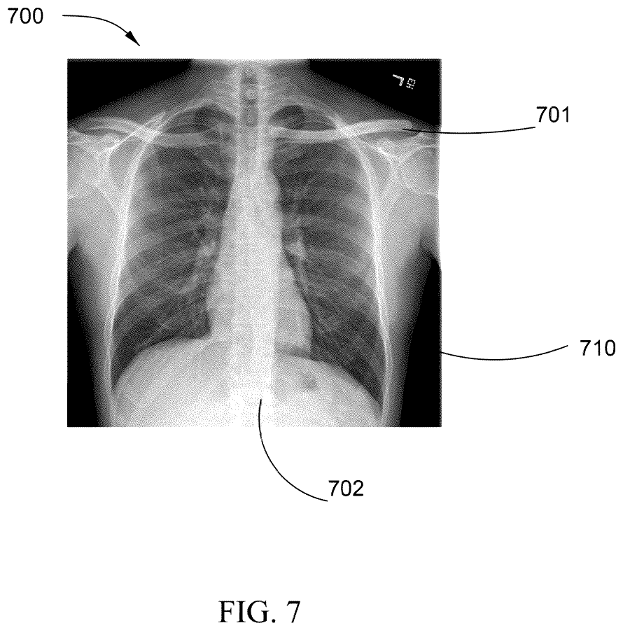

In some embodiments, the position of bone with abnormality may be displayed on a graphical user interface by means of an image or text bearing a position label. Specifically, the judgment unit or the image processing unit is capable of outputting an X-ray image bearing an indication sign, wherein the indication sign indicates, on the X-ray image, the position of bone where osteoporosis might be present. FIG. 7 shows a schematic diagram of an X-ray image bearing an indication sign according to some embodiments of the present application. As shown in FIG. 7, the X-ray image 710 may be any one of at least one image inputted to the image processing unit, and at least one indication sign, for example, including a first sign 701 at a position of scapula and a second sign 702 at a position of lumbar vertebra, is simultaneously displayed on the X-ray image 710. The at least one indication sign may, in any form, highlight different positions of bone. For example, the position of bone may be marked by means of an indication box, or may be filled with a color, or may be displayed by means of a mask, etc.

In some embodiments, when there are multiple positions of bone with abnormality, there may be a number of indication signs, and the judgment unit can use different kinds of label, or labels of different colors, to indicate different degrees of abnormality. Specifically, taking FIG. 7 as an example, when the degree of osteoporosis at scapula is high, the position of scapula may be indicated using a color with a high degree of saturation, and when the degree of abnormality at a lumbar vertebra site is low, the specific position of lumbar vertebra may be indicated using a color with a low degree of saturation or a different color. FIG. 7 merely serves as an example and does not constitute any limitation to the scope of the present application.

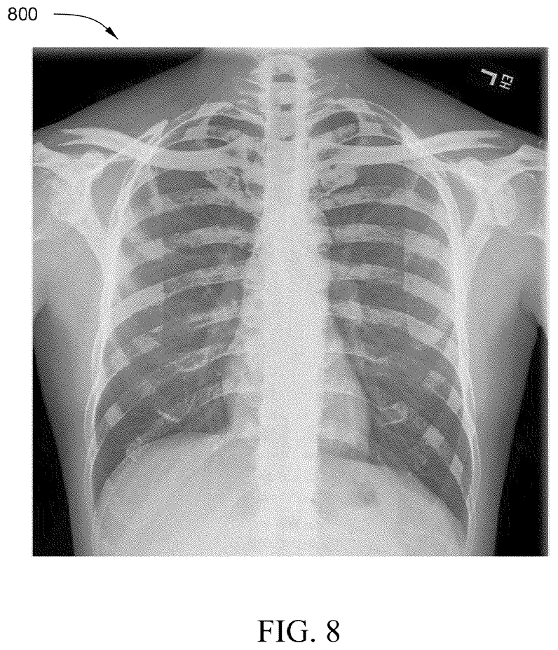

In some embodiments, in addition to the X-ray image bearing an indication sign shown in FIG. 7, other types of indicated image may be outputted. For example, FIG. 8 illustrates a schematic diagram of an X-ray image 800 bearing an indication sign according to some other embodiments of the present application. As shown in FIG. 8, the positions of bone with abnormality may be displayed on a graphical user interface by means of an infrared image, and different degrees of abnormality may be displayed by means of different colors or different degrees of saturation of a color. Of course, the position of bone with abnormality may also be displayed by means of other types of heat map.

In some other embodiments, the judgment unit may also indicate the position of bone by outputting the bone name or bone sign, among other forms, for the position of bone with abnormality, for example, outputting “the fourth joint of the lumbar vertebra” or “L4”, etc.

In some embodiments, the probability of abnormality may be an overall probability calculated for all bones in the X-ray image of the subject under examination, or may be a separate probability calculated for a position of bone with abnormality. The probability of abnormality may be indicated in the same manner or form as the position of bone with abnormality, or may be separately displayed to the user. For example, when the position of bone is shown in the manner as it is shown in the image of FIG. 7 or FIG. 8, the probability may be shown by means of a text label adjacent to the position of bone, or may be shown at a fixed place in the image. When the position of bone is shown in the form of a bone name or bone sign, the probability may be shown after the bone name, e.g., left scapula, 60%, etc.

In some embodiments, by the prompt of abnormality it is meant that when the T-score or classification of bone density of the subject under examination exceeds a threshold value range, the judgment unit or the image processing unit is capable of sending a prompt to the user or physician in various prompt forms such as a text or sound, for example, by displaying a pop-up window or a prompt icon on a graphical user interface, or playing an alert sound, etc.

In some embodiments, the image processing unit 320 further includes an identification unit 321. Specifically, the identification unit 321 is capable of identifying at least one region of interest of the X-ray image, and the classification unit 322 performs classification processing on the at least one region of interest to output a classification of bone density. Further, the identification unit 321 is capable of being further used to segment the at least one region of interest. Specifically, the at least one region of interest may include a bone of the subject under examination, i.e., the identification unit is capable of identifying and/or segmenting a bone portion of the subject under examination.

Specifically, the image processing unit 320 further includes an adjustment unit 325 configured to adjust the at least one region of interest on the basis of an input from a user, and perform classification processing on the basis of the adjusted region of interest. The adjustment unit 325 is provided to make it convenient for the user to manually adjust the region of interest or assign different weights to different regions of interest. For example, only the bone density of the lumbar vertebra portion is to be focused on, or the weight for the lumbar vertebra portion should exceed the weight for the scapula portion, which can facilitate subsequent classification or regression processing, etc.

Although the control apparatus is divided into multiple unit modules in the foregoing embodiments, it should be understood by those skilled in the art that the multiple unit modules can also be integrated. That is, the control apparatus of the X-ray imaging system according to some embodiments of the present application can be configured to execute: acquiring at least one X-ray image of a subject under examination, wherein the at least one X-ray image is a raw image or a medical image following image processing, and on the basis of a trained learning network, performing processing on the at least one X-ray image to output a result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

Specifically, the foregoing result of bone density and position of bone with abnormality, probability of abnormality and/or prompt of abnormality are all the outcomes of execution by or output from a computer. The result is only a probability value, and only serves to provide reference for a physician to accurately diagnose a disease and formulate a treatment scheme.

In some embodiments, the functions of the identification unit 321, the classification unit 322 (and/or the regression unit), and the judgment unit 323 are all implemented by the learning network. Of course, the identification unit and the classification unit may be configured to be implemented by the learning network, and the judgment unit may be a separate module integrated in the control apparatus. Of course, the identification unit may also be a separately configured module.

With further reference to FIG. 6, after inputting at least one X-ray image into the learning network 600, the learning network can output a T-score and/or classification of bone density, and position of bone with abnormality, probability of abnormality and/or prompt of abnormality.

In some embodiments, the information of the subject under examination is inputted into the learning network 600, and the at least one X-ray image is processed on the basis of the information of the subject under examination. The information of the subject under examination includes at least one of the age, gender, body weight, and exposure site of the subject under examination.

In some embodiments, the machine learning in the present application may include any suitable neural network model which is obtained by performing training using a clinical data set which includes clinical data of a plurality of subjects under examination, the clinical data of each subject under examination including an X-ray image and a corresponding T-score of bone density.

The learning network may include an input layer, an output layer, and a processing layer (or referred to as a hidden layer), wherein the input layer is configured to perform preprocessing, for example, deaveraging, normalization, or dimensionality reduction, on data or an image inputted, and the processing layer may include a convolutional layer for performing feature extraction, a batch normalization layer for performing standard normal distribution on the input, and an excitation layer for performing a nonlinear mapping on an output result of the convolutional layer. In addition, the learning network may further include a fully connected layer configured to output a corresponding offset.

Each convolutional layer includes several neurons, and the number of the neurons in each layer may be the same or set differently as required. On the basis of the first data set or the third data set (known input) and the second data set or the fourth data set (expected output), the number of processing layers in the network and the number of neurons in each processing layer are set, and a weight and/or a bias of the network is estimated (or adjusted or calibrated), so as to identify a mathematical relationship between the known input and the expected output and/or identify a mathematical relationship between the input and output of each layer.

Specifically, when the number of neurons in one of the layers is n, and the corresponding values of the n neurons are X₁, X₂, . . . and Xₙ; the number of neurons in the next layer connected to the one of the layers is m, and the corresponding values of the m neurons are Y₁, Y₂, . . . and Yₘ, then the two adjacent layers may be represented as:

$${Y}_{j} = f ( {\sum }_{i = 1}^{n} {W}_{ji} {X}_{i} + {B}_{j} )$$

where Xᵢ represents the value corresponding to the ith neuron of the upstream layer, Yⱼ represents the value corresponding to the jth neuron of the downstream layer, Wⱼᵢ represents the weight, and Bⱼ represents the bias. In some embodiments, the function f is a rectified linear function.

Thus, the mathematical relationship between the input and the output of each layer can be identified by adjusting the weight Wⱼᵢ and/or bias Bⱼ such that Loss Function converges, thereby obtaining the foregoing model through training.

In one embodiment, although the configuration of the learning network is guided by dimensions such as prior knowledge, input, and output of an estimation problem, optimal approximation of required output data is implemented depending on or exclusively according to input data. In various alternative implementations, clear meaning may be assigned to some data representations in the deep learning network using some aspects and/or features of data, an imaging geometry, a reconstruction algorithm, or the like, which helps to speed up training. This creates an opportunity to separately train (or pre-train) or define some layers in the learning network.

As discussed herein, deep learning technology (also referred to as deep machine learning, hierarchical learning, deep structured learning, and so on) employs an artificial neural network for learning. The deep learning method is characterized by using one or more network architectures to extract or simulate data of interest. The deep learning method may be implemented by using one or more processing layers (for example, an input layer, an output layer, a convolutional layer, a normalization layer, or a sampling layer, and processing layers of different numbers and functions may exist according to different deep network models), wherein the configuration and number of the layers allow a deep network to process complex information extraction and modeling tasks. Specific parameters (also referred to as “weight” or “bias”) of the network are usually estimated by means of a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. During image processing or reconstruction, this may be represented as different layers with respect to different feature levels in the data. Thus, processing may be performed layer by layer. That is, “simple” features may be correspondingly extracted from input data for an earlier or higher-level layer, and then these simple features are combined into a layer exhibiting features of higher complexity. In practice, each layer (or more specifically, each “neuron” in each layer) may process input data as output data for representation by using one or a plurality of linear and/or non-linear transformations (so-called activation functions). The number of the plurality of “neurons” may be constant among the plurality of layers or may vary from layer to layer. As discussed herein, as part of initial training of a deep learning process to solve a specific problem, a training data set includes a known input value (for example, a sample image or a pixel matrix of an image subjected to coordinate transformation) and an expected (target) output value (for example, an image or an identification and determination result) finally outputted in the deep learning process. In this manner, a deep learning algorithm can (in a supervised or guided manner or an unsupervised or unguided manner) process the training data set until a mathematical relationship between a known input and an expected output is identified and/or a mathematical relationship between the input and output of each layer is identified and represented. In the learning process, (a part of) input data is usually used, and a network output is created for the input data. Afterwards, the created network output is compared with the expected output of the data set, and then the difference between the created and expected outputs is used to iteratively update network parameters (weight and/or bias). A stochastic gradient descent (SGD) method may usually be used to update network parameters. However, those skilled in the art will appreciate that other methods known in the art may also be used to update network parameters. Similarly, a separate validation data set may be used to validate a trained network, wherein both a known input and an expected output are known. The known input is provided to the trained network so that a network output can be obtained, and then the network output is compared with the (known) expected output to validate prior training and/or prevent excessive training.

In some embodiments, the aforementioned trained network is obtained on the basis of training by a training module on an external carrier (for example, a device outside the medical imaging system). In some embodiments, the training system may include a first module configured to store a training data set, a second module configured to perform, on the basis of a model, training and/or updating, and a communication network configured to connect the first module and the second module. In some embodiments, the first module includes a first processing unit and a first memory cell, wherein the first memory cell is configured to store the training data set, and the first processing unit is configured to receive a relevant instruction (for example, acquire a training data set) and send the training data set according to the instruction. In addition, the second module includes a second processing unit and a second memory cell, wherein the second memory cell is configured to store a training model, and the second processing unit is configured to receive a relevant instruction and perform training and/or update of the network. In some other embodiments, the training data set may further be stored in the second memory cell of the second module, and the training system may not include the first module. In some embodiments, the communication network may include various connection types, such as wired or wireless communication links, or fiber-optic cables.

Once data (for example, a trained model) is generated and/or configured, the data can be replicated and/or loaded into the X-ray imaging system 100/200, which may also be accomplished in a different manner. For example, the model may be loaded by means of a directional connection or link between the X-ray imaging system 100/200 and the control unit. In this regard, communication between different elements may be accomplished using an available wired and/or wireless connection and/or according to any suitable communication (and/or network) standard or protocol. Alternatively or additionally, the data may be indirectly loaded into the X-ray imaging system 100/200. For example, the data may be stored in a suitable machine-readable medium (for example, a flash memory card), and then the medium is used to load the data into the X-ray imaging system 100/200 (for example, by a user or an authorized person of the system on site); or the data may be downloaded to an electronic device (for example, a notebook computer) capable of local communication, and then the device is used on site (for example, by the user or authorized person of the system) to upload the data to the X-ray imaging system 100/200 via a direct connection (for example, a USB connector).

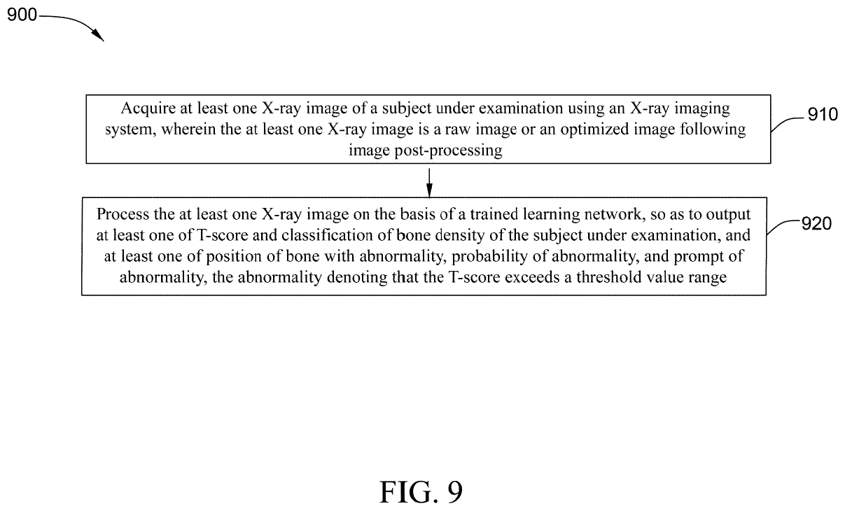

FIG. 9 is a flow chart of an X-ray image-based method for acquiring bone density according to some embodiments of the present invention. As shown in FIG. 9, the method 900 for acquiring bone density includes step 910 and step 920.

In step 910, at least one X-ray image of a subject under examination is acquired using an X-ray imaging system, wherein the at least one X-ray image is a raw image or a medical image following image processing.

Specifically, the X-ray image includes at least one or a combination of a single X-ray image, a dual-energy X-ray image, a stitched image, a tomographic (TOMO) image, or a combination of at least one of the foregoing and a low-dose local image. Specifically, what is acquired in step 910 may be a single raw X-ray image, or may be a medical image following image post-processing. Of course, what is acquired may be two raw images acquired using different exposure doses, or may be a medical image following dual-energy processing. Of course, what is acquired may be raw images of multiple X-ray sub-images to be stitched, or may be a stitched image following stitching, etc.

In step 920, the at least one X-ray image is processed on the basis of the trained learning network to output a result of the bone density of the subject under examination, and at least one of position of bone with abnormality, probability of abnormality, and prompt of abnormality, the result including at least one of T-score and classification of bone density, and the abnormality denoting that the result exceeds a threshold value range.

Specifically, step 920 further comprises performing classification processing on the at least one X-ray image to output a classification of bone density; and/or performing regression processing on the at least one X-ray image to output a T-score of bone density.

In some embodiments, the outputted T-score of bone density is a specific numerical value. The classifications of bone density may be first class, second class, and third class in a Chinese context, or may be level 1, level 2, and level 3 in an English context, or, of course, may be in any other suitable form of representation.

The abnormality denotes that the result of bone density exceeds a threshold value range, and specifically, may be the T-score of bone density exceeding a set threshold value or the classification of bone density exceeding a set limit. Specifically, when the result of bone density exceeds the threshold value range, it is indicated that there is a possibility of osteoporosis or a high probability of osteoporosis at the site in question. Of course, different threshold value ranges may be set to output different types of abnormality or degrees of abnormality. In some embodiments, different threshold value ranges may also be set according to information of the subject under examination, for example, according to age or gender.

The outputting position of bone with abnormality includes outputting an image or text bearing a position label, and specifically includes outputting an X-ray image, an infrared image, or other types of heat map bearing an indication sign, or the bone name or bone sign for the position of bone with abnormality. When there are multiple positions of bone with abnormality, there may be a number of indication signs, and different kinds or different colors or different degrees of saturation of a color can be used to perform labeling or displaying, so as to show different degrees of abnormality. The image or text can be shown by means of a graphical user interface of the display unit, and may also be shown together with the result of the bone density of the subject under examination.

The probability of abnormality may be an overall probability calculated for all bones in the X-ray image of the subject under examination, or may be a separate probability calculated for a position of bone with abnormality. The probability of abnormality may be shown in the same manner or form as the position of bone with abnormality, or may be separately displayed to the user. In some preferred embodiments, the probability of abnormality is generally simultaneously displayed on an image bearing a position label, for example, in the vicinity of bone bearing a label, so as to label the probability of abnormality at the position.

By the prompt of abnormality it is meant that when the T-score or classification of bone density of the subject under examination exceeds a threshold value range, a prompt is sent to the user or physician in various prompt forms such as a text or sound.

Specifically, step 920 further includes inputting information of the subject under examination into the trained learning network, and processing the at least one X-ray image on the basis of the information of the subject under examination. Specifically, the information of the subject under examination includes at least one of the age, gender, body weight, and exposure site of the subject under examination.

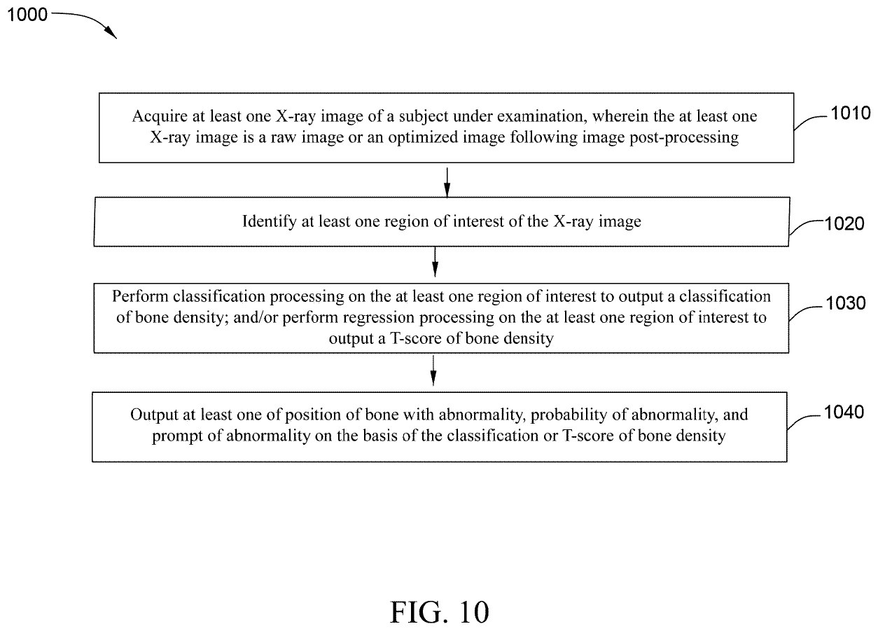

FIG. 10 is a flow chart of an X-ray image-based method for acquiring bone density according to some other embodiments of the present invention. As shown in FIG. 10, the method 1000 for acquiring bone density includes step 1010, step 1020, step 1030, and step 1040.

In step 1010, at least one X-ray image of a subject under examination is acquired using an X-ray imaging system, wherein the at least one X-ray image is a raw image or a medical image following image processing.

In step 1020, at least one region of interest of the X-ray image is identified.

Specifically, the at least one region of interest may include a bone of the subject under examination, i.e., the identification unit is capable of identifying and/or segmenting a bone portion of the subject under examination.

In some embodiments, step 1020 further includes segmenting the at least one region of interest. In some embodiments, step 1020 further includes adjusting the at least one region of interest on the basis of an input from a user.

In step 1030, classification processing is performed on the at least one region of interest to output a classification of bone density; and/or regression processing is performed on the at least one region of interest to output a T-score of bone density.

In step 1040, at least one of position of bone with abnormality, probability of abnormality, and prompt of abnormality is outputted on the basis of the classification or T-score of bone density.

The method for acquiring bone density according to some embodiments provided in the present invention is, firstly, capable of measuring or acquiring bone density on the basis of an X-ray image having been obtained by means of image capturing. On the one hand, a particular bone density measuring instrument is not required to perform examination, and on the other hand, the subject under examination does not have to receive additional radiation exposure. Secondly, the method, provided with a learning network, is capable of acquiring corresponding classification and T-score of bone density, and various possible positions, probabilities, or prompts of abnormality, so as to prompt a user in a visualized and direct manner.

An exemplary embodiment of the present invention provides an X-ray image-based method for acquiring bone density. The method for acquiring bone density includes acquiring at least one X-ray image of a subject under examination using an X-ray imaging system, wherein the at least one X-ray image is a raw image or a medical image following image processing, and on the basis of a trained learning network, outputting the result of the bone density of the subject under examination, and at least one of position of bone with abnormality, probability of abnormality, and prompt of abnormality, the result including at least one of T-score and classification of bone density, and the abnormality denoting that the result exceeds a threshold value range.

Specifically, the X-ray image includes at least one or a combination of a single X-ray image, a dual-energy X-ray image, a stitched image, a tomographic (TOMO) image, or a combination of at least one of the foregoing and a low-dose local image.

Specifically, the method for acquiring bone density further includes inputting information of the subject under examination into the trained learning network, and processing the at least one X-ray image on the basis of the information of the subject under examination.

Specifically, the information of the subject under examination includes at least one of the age, gender, body weight, and exposure site of the subject under examination.

Specifically, processing the at least one X-ray image on the basis of a trained learning network includes performing classification processing on the at least one X-ray image to output a classification of bone density; and/or performing regression processing on the at least one X-ray image to output a T-score of bone density.

Specifically, processing the at least one X-ray image further includes identifying at least one region of interest of the at least one X-ray image and performing classification and/or regression processing on the at least one region of interest.

Specifically, processing the at least one X-ray image further includes: adjusting the at least one region of interest on the basis of an input from a user, and performing classification and/or regression processing on the basis of the adjusted region of interest.

Specifically, the outputting the position of bone with abnormality includes outputting an X-ray image bearing a position label, wherein the indication sign indicates, on the X-ray image, the position of bone with abnormality.

Specifically, the X-ray image bearing a position label includes different kinds of label, or labels of different colors, so as to indicate different degrees of abnormality.

An exemplary embodiment of the present invention further provides an X-ray imaging system. The X-ray imaging system includes a control apparatus which is capable of executing the foregoing method for acquiring bone density.

An exemplary embodiment of the present invention further provides an X-ray imaging system. The X-ray imaging system includes an acquisition unit and an image processing unit. The acquisition unit is capable of acquiring at least one X-ray image of a subject under examination using the X-ray imaging system, wherein the at least one X-ray image is a raw image or a medical image following image processing, and the image processing unit is capable of, on the basis of a trained learning network, outputting the result of the bone density of the subject under examination, and at least one of position of bone with abnormality, probability of abnormality, and prompt of abnormality, the result including at least one of T-score and classification of bone density, and the abnormality denoting that the result exceeds a threshold value range.

Specifically, the X-ray image includes at least one or a combination of a single X-ray image, a dual-energy X-ray image, a stitched image, a tomographic (TOMO) image, or a combination of at least one of the foregoing and a low-dose local image.

Specifically, the learning network further processes the at least one X-ray image according to information of the subject under examination. Specifically, the information of the subject under examination includes at least one of the age, gender, body weight, and exposure site of the subject under examination.

Specifically, the image processing unit includes a classification unit and/or a regression unit, and a judgment unit, wherein the classification unit is configured to perform classification processing on the at least one X-ray image to output a classification of bone density, the regression unit is configured to perform regression processing on the at least one X-ray image to output a T-score of bone density, and the judgment unit is configured to output at least one of position of bone with abnormality, probability of abnormality, and prompt of abnormality, the abnormality denoting that the result exceeds a threshold value range.

Specifically, the image processing unit further includes an identification unit configured to identify at least one region of interest of the at least one X-ray image, and the classification unit and/or the regression unit perform(s) classification and/or regression processing on the at least one region of interest. Specifically, the image processing unit further includes an adjustment unit configured to adjust the at least one region of interest on the basis of an input from a user.

Specifically, the judgment unit is capable of outputting an X-ray image bearing a position label, wherein the indication sign indicates, on the X-ray image, the position of bone with abnormality. Specifically, the X-ray image bearing a position label includes different kinds of label, or labels of different colors, so as to indicate different degrees of abnormality.

The present invention may further provide a non-transitory computer-readable storage medium for storing an instruction set and/or a computer program. When executed by a computer, the instruction set and/or computer program causes the computer to execute the image processing distribution method. The computer executing the instruction set and/or computer program may be a computer of a medical imaging system, or may be other apparatuses/modules of the medical imaging system. In one embodiment, the instruction set and/or computer program may be programmed into a processor/control apparatus of the computer.

Specifically, when executed by the computer, the instruction set and/or computer program causes the computer to:
acquire at least one X-ray image of a subject under examination, wherein the at least one X-ray image is a raw image or a medical image following image processing; and
process the at least one X-ray image on the basis of a trained learning network to output a result of the bone density of the subject under examination, and at least one of position of bone with abnormality, probability of abnormality, and prompt of abnormality, the result including at least one of T-score and classification of bone density, and the abnormality denoting that the result exceeds a threshold value range.

The instructions described above may be combined into one instruction for execution, and any of the instructions may also be split into a plurality of instructions for execution. Moreover, the present invention is not limited to the instruction execution order described above.

As used herein, the term “computer” may include any processor-based or microprocessor-based system, including a system using a microcontrol apparatus, a reduced instruction set computer (RISC), an application-specific integrated circuit (ASIC), a logic circuit, and any other circuit or processor capable of executing the functions described herein. The examples above are exemplary only and are not intended to limit the definition and/or meaning of the term “computer” in any way.

Some exemplary embodiments have been described above; however, it should be understood that various modifications may be made. For example, suitable results can be achieved if the described techniques are executed in a different order and/or if components in the described systems, architectures, devices, or circuits are combined in different ways and/or replaced or supplemented by additional components or equivalents thereof. Accordingly, other implementations also fall within the scope of protection of the claims.

1. An X-ray image-based method for acquiring bone density, comprising:
acquiring at least one X-ray image of a subject under examination using an X-ray imaging system, wherein the at least one X-ray image is a raw image or a medical image following image processing; and
processing the at least one X-ray image on the basis of a trained learning network to output a result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

2. The method for acquiring bone density according to claim 1, wherein the X-ray image comprises at least one or a combination of a single X-ray image, a dual-energy X-ray image, a stitched image, and a tomographic (TOMO) image, or a combination of at least one of the foregoing and a low-dose local image.

3. The method for acquiring bone density according to claim 1, wherein the method further comprises: inputting information of the subject under examination into the trained learning network, and processing the at least one X-ray image on the basis of the information of the subject under examination.

4. The method for acquiring bone density according to claim 3, wherein the information of the subject under examination comprises at least one of the age, gender, body weight, and exposure site of the subject under examination.

5. The method for acquiring bone density according to claim 1, wherein processing the at least one X-ray image on the basis of a trained learning network comprises:
performing classification processing on the at least one X-ray image to output a classification of bone density; and/or performing regression processing on the at least one X-ray image to output a T-score of bone density.

6. The method for acquiring bone density according to claim 5, wherein processing the at least one X-ray image further comprises:
identifying at least one region of interest of the at least one X-ray image and performing classification and/or regression processing on the at least one region of interest.

7. The method for acquiring bone density according to claim 6, wherein processing the at least one X-ray image further comprises: adjusting the at least one region of interest on the basis of an input from a user, and performing classification and/or regression processing on the basis of the adjusted region of interest.

8. The method for acquiring bone density according to claim 1, wherein outputting the position of bone with abnormality comprises outputting an X-ray image bearing a position label, wherein the position label indicates, on the X-ray image, the position of bone with abnormality.

9. The method for acquiring bone density according to claim 8, wherein the X-ray image bearing a position label comprises different kinds of label, or labels of different colors, so as to indicate different degrees of abnormality.

10. An X-ray imaging system, comprising:
an acquisition unit configured to acquire at least one X-ray image of a subject under examination using an X-ray imaging system, wherein the at least one X-ray image is a raw image or a medical image following image processing; and
an image acquisition unit configured to process the at least one X-ray image on the basis of a trained learning network to output a result of the bone density of the subject under examination, and at least one of position of bone with abnormality, probability of abnormality, and prompt of abnormality, the result comprising at least one of T-score and classification of bone density, and the abnormality denoting that the result exceeds a threshold value range.

11. The X-ray imaging system according to claim 10, wherein the at least one X-ray image comprises at least one or a combination of a single X-ray image, a dual-energy X-ray image, a stitched image, and a tomographic (TOMO) image, or a combination of at least one of the foregoing and a low-dose local image.

12. The X-ray imaging system according to claim 10, wherein the image acquisition unit is further configured to input information of the subject under examination into the trained learning network, and process the at least one X-ray image on the basis of the information of the subject under examination.

13. The X-ray imaging system according to claim 12, wherein the information of the subject under examination comprises at least one of the age, gender, body weight, and exposure site of the subject under examination.

14. The X-ray imaging system according to claim 10, wherein processing the at least one X-ray image on the basis of a trained learning network comprises:
performing classification processing on the at least one X-ray image to output a classification of bone density; and/or performing regression processing on the at least one X-ray image to output a T-score of bone density.

15. The X-ray imaging system according to claim 14, wherein processing the at least one X-ray image further comprises:
identifying at least one region of interest of the at least one X-ray image and performing classification and/or regression processing on the at least one region of interest.

16. The X-ray imaging system according to claim 15, wherein processing the at least one X-ray image further comprises: adjusting the at least one region of interest on the basis of an input from a user, and performing classification and/or regression processing on the basis of the adjusted region of interest.

17. The X-ray imaging system according to claim 10, wherein outputting the position of bone with abnormality comprises outputting an X-ray image bearing a position label, wherein the position label indicates, on the X-ray image, the position of bone with abnormality.

18. The X-ray imaging system according to claim 17, wherein the X-ray image bearing a position label comprises different kinds of label, or labels of different colors, so as to indicate different degrees of abnormality.
